SEQUENCE LISTING (22 sequences)

SEQ ID NO: 1 - DNA, 30 bp, Artificial  sequence
  Single strand DNA oligonucleotide
gaggtgggctggtatcgcagtccgtttgag

SEQ ID NO: 2 - DNA, 38 bp, Artificial  sequence
  Single strand DNA oligonucleotide
gatcctcaaacggactgcgataccagcccacctctgca

SEQ ID NO: 3 - DNA, 42 bp, Artificial  sequence
  Single strand DNA oligonucleotide
agctttgccgaggtgggctggtatcgcagtccgtttattgca

SEQ ID NO: 4 - DNA, 34 bp, Artificial  sequence
  Single strand DNA oligonucleotide
ataaacggactgcgataccagcccacctcggcaa

SEQ ID NO: 5 - DNA, 19 bp, Artificial  sequence
  Single strand DNA oligonucleotide
cagcttgctttcgagatga

SEQ ID NO: 6 - DNA, 19 bp, Artificial  sequence
  Single strand DNA oligonucleotide
agtagcagaagcctgaaga

SEQ ID NO: 7 - protein, 218 aa, Mus musculus
GQFRVIGPGYPIRALVGDEAELPCRISPGKNATGMEVGWYRSPFSRVVHLYRNGKDQDAEQAPEYRERTELLKETISEGKVTLRIQNVRFSDEGGYTCFFRDHSYQEEAAMELKVEDPFYWVNPGVLTLIALVPTILLQVPVGLVFLFLQHRLRGKLRAEVENLHRTFDPHFLRVPCWKITLFVIVPVLGPLVALIICYNWLHRRLAGQFLEELRNPF

SEQ ID NO: 8 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 9 - protein, 21 aa, Artificial  sequence
  MOG 35-55 epitope polypeptide
MEVGWYRSPFSRVVHLYRNGK

SEQ ID NO: 10 - protein, 8 aa, Artificial  sequence
  Mouse MOG 37-44 polypeptide
VGWYRSPF

SEQ ID NO: 11 - DNA, 9183 bp, Artificial  sequence
  Filamentous phage display vector f8-1
aacgctactaccattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgaactacagcaccagattcagcaattaagctctaagccatccgcaaaaatgacctcttatcaaaaggagcaattaaaggtactgtctaatcctgacctgttggaatttgcttccggtctggttcgctttgaggctcgaattgaaacgcgatatttgaagtctttcgggcttcctcttaatctttttgatgcaattcgctttgcttctgactataatagacagggtaaagacctgatttttgatttatggtcattctcgttttctgaactgtttaaagcatttgagggggattcaatgaatatttatgacgattccgcagtattggacgctatccagtctaaacattttacaattaccccctctggcaaaacttcctttgcaaaagcctctcgctattttggtttctatcgtcgtctggttaatgagggttatgatagtgttgctcttaccatgcctcgtaattccttttggcgttatgtatctgcattagttgagtgtggtattcctaaatctcaattgatgaatctttccacctgtaataatgttgttccgttagttcgttttattaacgtagatttttcctcccaacgtcctgactggtataatgagccagttcttaaaatcgcataaggtaattcaaaatgattaaagttgaaattaaaccgtctcaagcgcaatttactacccgttctggtgtttctcgtcagggcaagccttattcactgaatgagcagctttgttacgttgatttgggtaatgaatatccggtgcttgtcaagattactctcgacgaaggtcagccagcgtatgcgcctggtctgtacaccgtgcatctgtcctcgttcaaagttggtcagttcggttctcttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctccgtagccgttgctaccctcgttccgatgctgtctttcgctgcagagggtgaggatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaagatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaatcatgccagttcttttgggtattccgttattattgcgtttcctcggtttccttctggtaactttgttcggctatctgcttactttccttaaaaagggcttcggtaagatagctattgctatttcattgtttcttgctcttattattgggcttaactcaattcttgtgggttatctctctgatattagcgcacaattaccctctgattttgttcagggcgttcagttaattctcccgtctaatgcgcttccctgtttttatgttattctctctgtaaaggctgctattttcatttttgacgttaaacaaaaaatcgtttcttatttggattgggataaataaatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatttctgatttgcttgctattggtcgtggtaatgattcctacgacgaaaataaaaacggtttgcttgttcttgatgaatgcggtacttggtttaatacccgttcatggaatgacaaggaaagacagccgattattgattggtttcttcatgctcgtaaattgggatgggatattatttttcttgttcaggatttatctattgttgataaacaggcgcgttctgcattagctgaacacgttgtttattgtcgccgtctggacagaattactttaccctttgtcggcactttatattctcttgttactggctcaaaaatgcctctgcctaaattacatgttggtgttgttaaatatggtgattctcaattaagccctactgttgagcgttggctttatactggtaagaatttatataacgcatatgacactaaacaggctttttccagtaattatgattcaggtgtttattcatatttaaccccttatttatcacacggtcggtatttcaaaccattaaatttaggtcagaagatgaaattaactaaaatatatttgaaaaagttttctcgcgttctttgtcttgcgataggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattccatcacatatattgatttatgtactgtttcaattaaaaaaggtaattcaaatgaaattgttaaatgtaattaattttgttttcttgatgtttgtttcatcatcttcttttgctcaagtaattgaaatgaataattcgcctctgcgcgatttcgtgacttggtattcaaagcaaacaggtgaatctgttattgtctcacctgatgttaaaggtacagtgactgtatattcctctgacgttaagcctgaaaatttacgcaatttctttatctctgttttacgtgctaataattttgatatggttggctcaattccttccataattcagaaatataacccaaatagtcaggattatattgatgaattgccatcatctgatattcaggaatatgatgataattccgctccttctggtggtttctttgttccgcaaaatgataatgttactcaaacatttaaaattaataacgttcgcgcaaaggatttaataagggttgtagaattgtttgttaaatctaatacatctaaatcctcaaatgtattatctgttgatggttctaacttattagtagttagcgcccctaaagatattttagataaccttccgcaatttctttctactgttgatttgccaactgaccagatattgattgaaggattaattttcgaggttcagcaaggtgatgctttagatttttcctttgctgctggctctcagcgcggcactgttgctggtggtgttaatactgaccgtctaacctctgttttatcttctgcgggtggttcgttcggtatttttaacggcgatgttttagggctatcagttcgcgcattaaagactaatagccattcaaaaatattgtctgtgcctcgtattcttacgctttcaggtcagaagggttctatttctgttggccagaatgtcccttttattactggtcgtgtaactggtgaatctgccaatgtaaataatccatttcagacggttgagcgtcaaaatgttggtatttctatgagtgtttttcccgttgcaatggctggcggtaatattgttttagatataaccagtaaggccgatagtttgagttcttctactcaggcaagtgatgttattactaatcaaagaagtattgcgacaacggttaatttgcgtgatggtcagactcttttgctcggtggcctcactgattacaaaaacacttctcaagattctggtgtgccgttcctgtctaaaatccctttaatcggcctcctgtttagctcccgttctgattctaacgaggaaagcacgttgtacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttctccggctttccccgtcaagctctaaatcgggggatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaattagccttgaatacattactggtaaggtaaacgccattgtcagcaaattgatccaagagaaccaacttaaagcttatgatgatgatgtgcttaaaaacttactcaatggctggtttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgccccatttagtggctattcttcctgccacaaaggcttggaatactgagtgtaaaagaccaagacccgctaatgaaaagccaaccatcatgctattccatccaaaacgattttcggtaaatagcacccacaccgttgcgggaatttggcctatcaattgcgctgaaaaataaataatcaacaaaatgggcatcgttttaaataaagtgatgtataccgaattcgattgcgtctcaacccctacttcggtatctgtattatcacgtgtatttttggtttcacggaaccaaaacataaccacaaggaaagtgacaatatttagcaacgcagcgataaaaaagggactatgcggtgaaatctctcctgcaaaaccaccaataataggccccgctattaaaccaagcccaaaacttgcccctaaccaaccgaaccacttcacgcgttgagaagctgaggtggtatcggcaatgaccgatgccgcgacagccccagtagctcctgtgatccctgaaagcaaacggcctaaatacagcatccaaagcgcacttgaaaaagccagcaataagtaatccagcgatgcgcctattaatgacaacaacagcactgggcgccgaccaaatcggtcagacatttttccaagccaaggagcaaagataacctgcattaacgcataaagtgcaagcaatacgccaaagtggttagcgatatcttccgaagcaataaattcacgtaataacgttggcaagactggcatgataaggccaatccccatggcatcgagtaacgtaattaccaatgcgatctttgtcgaactattcatttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttaaccaccctttagattcataaagcgaaaataatgcggctccaacgtacccacctaaatggaaacggcgttcactccaatctaaacacgcacaacagattttacgtgaatgtttggaaggaacgtcaattcccatttcatgaaaatattgaataccacttaatgtgatcattgaaccattttcagtgatccattgctgttgacaaagggaatcatagatccctttagggttccgatttagtgctttacggcacctcgacctccaaaaacttgatttgggtgatggttcacgtagtgggccatcgccctgatagacggtttttcgccctttgacgttggagtccacgttctttaatagtggactcttgttccaaactggaacaacactcacaactaactcggcctattcttttgatttataaggatttttgtcattttctgcttactggttaaaaaataagctgatttaacaaatatttaacgcgaaatttaacaaaacattaacgtttacaatttaaatatttgcttatacaatcatcctgtttttggggcttttctgattatcaatcggggtacatatgattgacatgctagttttacgattaccgttcatcgattctcttgtttgctccagactttcaggtaatgacctgatagcctttgtagacctctcaaaaatagctaccctctccggcatgaatttatcagctagaacggttgaatatcatattgacggtgatttgactgtctccggcctttctcacccgtttgaatctttgcctactcattactccggcattgcatttaaaatatatgagggttctaaaaatttttatccctgcgttgaaattaaggcttcaccagcaaaagtattacagggtcataatgtttttggtacaaccgatttagctttatgctctgaggctttattgcttaattttgctaactctctgccttgcttgtacgatttattggatgtt

SEQ ID NO: 12 - DNA, 24 bp, Artificial  sequence
  Mouse MOG 37-44 peptide coding sequence
gtgggctggtatcgcagtccgttt

SEQ ID NO: 13 - protein, 81 aa, Homo sapiens
MSQKPAKEGPRLSKNQKYSEHFSIHCCPPFTFLNSKKEIVDRKYSICKSGCFYQKKEEDWICCACQKTRLKRKIRPTPKKK

SEQ ID NO: 14 - protein, 207 aa, Homo sapiens
MVATCLQVVGFVTSFVGWIGVIVTTSTNDWVVTCGYTIPTCRKLDELGSKGLWADCVMATGLYHCKPLVDILILPGYVQACRALMIAASVLGLPAILLLLTVLPCIRMGQEPGVAKYRRAQLAGVLLILLALCALVATIWFPVCAHRETTIVSFGYSLYAGWIGAVLCLVGGCVILCCAGDAQAFGENRFYYTAGSSSPTHAKSAHV

SEQ ID NO: 15 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 16 - protein, 582 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESKEVSTLESH

SEQ ID NO: 17 - DNA, 82 bp, Artificial  sequence
  Partial sequence of MOG f8
gttgcgactcttgttcctatgctaagctttgccgaggtgggctggtatcgcagtccgtttattgcagaaggtgatgacccgg

SEQ ID NO: 18 - protein, 200 aa, Homo sapiens
GQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFESFGVLGPQVKEPKKTGQFLEELLFHLEALSG

SEQ ID NO: 19 - protein, 8 aa, Artificial  sequence
  Human MOG 37-44 polypeptide
VGWYRPPF

SEQ ID NO: 20 - DNA, 9234 bp, Artificial  sequence
  Filamentous phage display vector f88-4
aacgctactaccattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgaactacagcaccagattcagcaattaagctctaagccatccgcaaaaatgacctcttatcaaaaggagcaattaaaggtactgtctaatcctgacctgttggaatttgcttccggtctggttcgctttgaggctcgaattgaaacgcgatatttgaagtctttcgggcttcctcttaatctttttgatgcaattcgctttgcttctgactataatagacagggtaaagacctgatttttgatttatggtcattctcgttttctgaactgtttaaagcatttgagggggattcaatgaatatttatgacgattccgcagtattggacgctatccagtctaaacattttacaattaccccctctggcaaaacttcctttgcaaaagcctctcgctattttggtttctatcgtcgtctggttaatgagggttatgatagtgttgctcttaccatgcctcgtaattccttttggcgttatgtatctgcattagttgagtgtggtattcctaaatctcaattgatgaatctttccacctgtaataatgttgttccgttagttcgttttattaacgtagatttttcctcccaacgtcctgactggtataatgagccagttcttaaaatcgcataaggtaattcaaaatgattaaagttgaaattaaaccgtctcaagcgcaatttactacccgttctggtgtttctcgtcagggcaagccttattcactgaatgagcagctttgttacgttgatttgggtaatgaatatccggtgcttgtcaagattactctcgacgaaggtcagccagcgtatgcgcctggtctgtacaccgtgcatctgtcctcgttcaaagttggtcagttcggttctcttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctccgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaatcatgccagttcttttgggtattccgttattattgcgtttcctcggtttccttctggtaactttgttcggctatctgcttactttccttaaaaagggcttcggtaagatagctattgctatttcattgtttcttgctcttattattgggcttaactcaattcttgtgggttatctctctgatattagcgcacaattaccctctgattttgttcagggcgttcagttaattctcccgtctaatgcgcttccctgtttttatgttattctctctgtaaaggctgctattttcatttttgacgttaaacaaaaaatcgtttcttatttggattgggataaataaatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatttctgatttgcttgctattggtcgtggtaatgattcctacgacgaaaataaaaacggtttgcttgttcttgatgaatgcggtacttggtttaatacccgttcatggaatgacaaggaaagacagccgattattgattggtttcttcatgctcgtaaattgggatgggatattatttttcttgttcaggatttatctattgttgataaacaggcgcgttctgcattagctgaacacgttgtttattgtcgccgtctggacagaattactttaccctttgtcggcactttatattctcttgttactggctcaaaaatgcctctgcctaaattacatgttggtgttgttaaatatggtgattctcaattaagccctactgttgagcgttggctttatactggtaagaatttatataacgcatatgacactaaacaggctttttccagtaattatgattcaggtgtttattcatatttaaccccttatttatcacacggtcggtatttcaaaccattaaatttaggtcagaagatgaaattaactaaaatatatttgaaaaagttttctcgcgttctttgtcttgcgataggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattccatcacatatattgatttatgtactgtttcaattaaaaaaggtaattcaaatgaaattgttaaatgtaattaattttgttttcttgatgtttgtttcatcatcttcttttgctcaagtaattgaaatgaataattcgcctctgcgcgatttcgtgacttggtattcaaagcaaacaggtgaatctgttattgtctcacctgatgttaaaggtacagtgactgtatattcctctgacgttaagcctgaaaatttacgcaatttctttatctctgttttacgtgctaataattttgatatggttggctcaattccttccataattcagaaatataacccaaatagtcaggattatattgatgaattgccatcatctgatattcaggaatatgatgataattccgctccttctggtggtttctttgttccgcaaaatgataatgttactcaaacatttaaaattaataacgttcgcgcaaaggatttaataagggttgtagaattgtttgttaaatctaatacatctaaatcctcaaatgtattatctgttgatggttctaacttattagtagttagcgcccctaaagatattttagataaccttccgcaatttctttctactgttgatttgccaactgaccagatattgattgaaggattaattttcgaggttcagcaaggtgatgctttagatttttcctttgctgctggctctcagcgcggcactgttgctggtggtgttaatactgaccgtctaacctctgttttatcttctgcgggtggttcgttcggtatttttaacggcgatgttttagggctatcagttcgcgcattaaagactaatagccattcaaaaatattgtctgtgcctcgtattcttacgctttcaggtcagaagggttctatttctgttggccagaatgtcccttttattactggtcgtgtaactggtgaatctgccaatgtaaataatccatttcagacggttgagcgtcaaaatgttggtatttctatgagtgtttttcccgttgcaatggctggcggtaatattgttttagatataaccagtaaggccgatagtttgagttcttctactcaggcaagtgatgttattactaatcaaagaagtattgcgacaacggttaatttgcgtgatggtcagactcttttgctcggtggcctcactgattacaaaaacacttctcaagattctggtgtgccgttcctgtctaaaatccctttaatcggcctcctgtttagctcccgttctgattctaacgaggaaagcacgttgtacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttctccggctttccccgtcaagctctaaatcgggggagctcgagcttactccccatccccctgttgacaattaatcatcggctcgtataatgtgtggaattgtgagcggataacaatttcttaatggaaacttcctcatgaaaaagtctttagttcttaaagcatctgttgctgttgcgactcttgttcctatgctaagctttgccaacgtccctgcagaaggtgatgacccggctaaagctgcttttgactctcttcaggcttctgctactgaatacatcggctacgcttgggctatggtggttgttatcgttggtgctactattggcatcaaacttttcaaaaaattcacttctaaagcgtcttaatgaactcagatacccagcccgcctaatgagcgggcttttttttaagctagcttatgatgatgatgtgcttaaaaacttactcaatggctggtttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgccccatttagtggctattcttcctgccacaaaggcttggaatactgagtgtaaaagaccaagacccgctaatgaaaagccaaccatcatgctattccatccaaaacgattttcggtaaatagcacccacaccgttgcgggaatttggcctatcaattgcgctgaaaaataaataatcaacaaaatgggcatcgttttaaataaagtgatgtataccgaattcgattgcgtctcaacccctacttcggtatctgtattatcacgtgtatttttggtttcacggaaccaaaacataaccacaaggaaagtgacaatatttagcaacgcagcgataaaaaagggactatgcggtgaaatctctcctgcaaaaccaccaataataggccccgctattaaaccaagcccaaaacttgcccctaaccaaccgaaccacttcacgcgttgagaagctgaggtggtatcggcaatgaccgatgccgcgacagccccagtagctcctgtgatccctgaaagcaaacggcctaaatacagcatccaaagcgcacttgaaaaagccagcaataagtaatccagcgatgcgcctattaatgacaacaacagcactgggcgccgaccaaatcggtcagacatttttccaagccaaggagcaaagataacctgcattaacgcataaagtgcaagcaatacgccaaagtggttagcgatatcttccgaagcaataaattcacgtaataacgttggcaagactggcatgataaggccaatccccatggcatcgagtaacgtaattaccaatgcgatctttgtcgaactattcatttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttaaccaccctttagattcataaagcgaaaataatgcggctccaacgtacccacctaaatggaaacggcgttcactccaatctaaacacgcacaacagattttacgtgaatgtttggaaggaacgtcaattcccatttcatgaaaatattgaataccacttaatgtgatcattgaaccattttcagtgatccattgctgttgacaaagggaatcatagatccctttagggttccgatttagtgctttacggcacctcgacctccaaaaacttgatttgggtgatggttcacgtagtgggccatcgccctgatagacggtttttcgccctttgacgttggagtccacgttctttaatagtggactcttgttccaaactggaacaacactcacaactaactcggcctattcttttgatttataaggatttttgtcattttctgcttactggttaaaaaataagctgatttaacaaatatttaacgcgaaatttaacaaaacattaacgtttacaatttaaatatttgcttatacaatcatcctgtttttggggcttttctgattatcaatcggggtacatatgattgacatgctagttttacgattaccgttcatcgattctcttgtttgctccagactttcaggtaatgacctgatagcctttgtagacctctcaaaaatagctaccctctccggcatgaatttatcagctagaacggttgaatatcatattgacggtgatttgactgtctccggcctttctcacccgtttgaatctttgcctactcattactccggcattgcatttaaaatatatgagggttctaaaaatttttatccctgcgttgaaattaaggcttcaccagcaaaagtattacagggtcataatgtttttggtacaaccgatttagctttatgctctgaggctttattgcttaattttgctaactctctgccttgcttgtacgatttattggatgtt

SEQ ID NO: 21 - DNA, 24 bp, Artificial  sequence
  MOG 37-44 epitope coding sequence
gtggggtggtaccgcccccccttc

SEQ ID NO: 22 - DNA, 77 bp, Artificial  sequence
  Partial sequence of MOG  f88
accctcgttccgatgctgtctttcgctgcagaggtgggctggtatcgcagtccgtttgaggatcccgcaaaagcggc

(19) United States
(12) Patent Application Publication
(10) Pub. No.: US 2009/0304726 A1
(43) Pub. Date: Dec. 10, 2009

(54) VIRAL DISPLAY VEHICLES FOR TREATING MULTIPLE SCLEROSIS

(75) Inventors: Beka Solomon, Herzlia Pituach (IL); Natalla Zabavnik, Rishon-Iezion (IL); Rela Koppel, Tel-aviv (IL); Idan Rakover, Herzlia (IL)

Correspondence Address: BROWDY AND NEIMARK, P.L.L.C.;624 NINTH STREET, NW, SUITE 300, WASHINGTON, DC, 20001-5303, US

(73) Assignee: RAMOT AT TEL AVIV UNIVERSITY LTD., TEL AVIV (IL)

(21) Appl. No.: 12/279,694

(22) PCT Filed: Feb. 15, 2007
(86) PCT No.: PCT/IL2007/000216

§ 371 Date: Jun. 25, 2009

Related U.S. Application Data
(60) Provisional application No. 60/773,351, filed on Feb. 15, 2006.

(51) Int. Cl. A61K 39/00

(52) U.S. Cl. 424/185.1; 424/184.1

(57) ABSTRACT

Provided are viral display vehicles which display multiple sclerosis associated antigens on the surface thereof for induction of immune tolerance to autoantigens such as MOG. Also provided are methods and pharmaceutical compositions for treating multiple sclerosis using the viral display vehicles of the present invention.

### FIELD AND BACKGROUND OF THE INVENTION

The present invention relates to viral display vehicles displaying multiple sclerosis associated autoantigens which can be used to induce tolerance against auto-antigens associated with multiple sclerosis, and more particularly, to administration of such viral display vehicles for treating multiple sclerosis.

Multiple sclerosis (MS) is a chronic inflammatory disease of the central nervous system (CNS) affecting young adults (disease onset between 20 to 40 years of age), and the third leading cause for disability after trauma and rheumatic diseases. Disease prevalence is 120/100,000 and there are currently between 250,000 to 350,000 cases in North America. MS is characterized by a prominent infiltration of macrophages and T lymphocytes through the blood brain barrier (BBB) which induces active inflammation within the brain and spinal cord, attacking the myelin and resulting in gliotic scars, axonal loss and demyelination in the brain and spinal cord. Acute and chronic inflammatory processes characteristics to MS are visualized by brain and spinal cord MRI as hyperintense T2 or hypointense T1 lesions.

The etiology of MS is not fully understood. The disease develops in genetically predisposed subjects exposed to yet undefined environmental factors and the pathogenesis involves autoimmune mechanisms associated with autoreactive T cells against myelin antigens. MS is subdivided into several clinical subtypes; first signs involve onset of neurological symptoms affecting the CNS and accompanied by demyelinating lesions on brain magnetic resonance imaging (MRI). In 85% of the patients the disease is characterized by a relapsing-remitting mode (RRMS). In about 15% of patients the disease has a primary progressive course, characterized by gradual onset of neurological symptoms that progress over time, without any attacks. The only course of MS in which treatment was effectively established is RRMS. Thus, various immunomodulatory drugs such as interferon beta-1a have been shown to reduce the number and severity of acute attacks, and decrease the accumulation of neurological disability. However, the overall efficacy of these drugs is limited to only 30-35% of the cases.

The experimental model for MS, allergic encephalomyelitis (EAE), supports an autoimmune mechanism. In that model autoimmunity is mediated by CD4+ T cells which can be induced experimentally in susceptible strains of laboratory animals by immunization with CNS antigens, such as myelin basic protein (MBP) or proteolipid protein (PLP), as well as by adoptive transfer of activated CD4+ T cells specific for myelin antigens in appropriate adjuvant.

Analysis of the T cell reactivity to myelin antigens in MS patients revealed that the autoimmune response to myelin oligodendrocyte glycoprotein (MOG) predominates that of MBP, PLP or myelin-associated glycoprotein (MAG), and is directed against three main MOG epitopes; amino acids 1-22, 34-56 and 64-96 (Kerlero de Rosbo et al. 1997). Therefore, MOG is widely recognized as an important potential target antigen in MS pathogenesis.

MOG is a minor component of CNS myelin, exposed on the surface of the outermost lamellae of the myelin sheath. It is a glycoprotein consisting of 218-amino-acids (GenBank Accession No. AAA03180) with an extracellular Ig-like domain encompassing amino acids 1-125. Recent studies demonstrated that MOG is strongly encephalogenic in mice (Amor et al. 1994). Sun D., et al., (2001) demonstrated that administration of MOG₃₅₋₅₅ peptide results in enrichment of CD8+ αβTCR+ T cells which when administered to mice cause encephalomyelitis via adoptive transfer. Sao H., et al. (2004) demonstrated the induction of optic neuritis (ON) and EAE by subcutaneous and footpad injections of MOG₃₅₋₅₅ and MOG₄₀₋₅₄ peptides.

Studies in experimental autoimmune models have shown the feasibility of inducing antigen-specific tolerance for disease resistance. Thus, intranasal administration of encephalitogenic epitopes of MBP was shown to protect against EAE induction (Bai et al. 1997). Other studies showed that antigen-specific tolerance is dependent on the route of administration and the amount of antigen used (Friedman and Weiner 1994). Thus, U.S. Pat. No. 5,645,820 (to Hafler D A. and Weiner H L.) discloses aerosol or oral administration of auto-antigens such as MBP and type II collagen for the treatment multiple sclerosis and arthritis, respectively. However, due to their limited effect on disease symptoms (e.g., only a slight decrease in EAE scores from 3-4 to 2), such immunization modes are not clinically practiced.

U.S. Pat. No. 6,703,015 (to Solomon B. and Frenkel D.) discloses a display vehicle presenting a beta-amyloid epitope for treating Alzheimer's disease. When administered intraperitoneally or intranasally to a subject, the display vehicle was capable of eliciting antibodies against the beta-amyloid epitope and thus treat Alzheimer's disease. However, since these viral display vehicles resulted in production of endogenous antibodies against the displayed epitope, such display vehicles were never suggested for treating multiple sclerosis or any other autoimmune disease where auto-antibodies are deleterious and undesired.

There is thus a widely recognized need for, and it would be highly advantageous to have, methods and compositions for treating multiple sclerosis devoid of the above limitations.

### SUMMARY OF THE INVENTION

According to one aspect of the present invention there is provided a composition-of-matter comprising a viral display vehicle displaying a multiple sclerosis associated antigen on a surface thereof.

According to another aspect of the present invention there is provided a pharmaceutical composition comprising, as an active ingredient, the composition-of-matter and a pharmaceutically acceptable carrier.

According to yet another aspect of the present invention there is provided a method of treating a multiple sclerosis, the method comprising administering to a subject in need thereof a therapeutically effective amount of the pharmaceutical composition, thereby treating the multiple sclerosis.

According to still another aspect of the present invention there is provided a use of the composition-of-matter for the manufacturing of a medicament identified for the treatment of multiple sclerosis.

According to further features in preferred embodiments of the invention described below, the pharmaceutically acceptable carrier is formulated for mucosal administration.

According to still further features in the described preferred embodiments administering is effected by trans-mucosal administration.

According to still further features in the described preferred embodiments administering is effected by intranasal administration.

According to still further features in the described preferred embodiments the medicament is formulated for trans-mucosal administration.

According to still further features in the described preferred embodiments the medicament is formulated for intranasal administration.

According to still further features in the described preferred embodiments the multiple sclerosis associated antigen comprises a MOG antigen.

According to still further features in the described preferred embodiments the MOG antigen comprises amino acids 37-44 of SEQ ID NO: 18.

According to still further features in the described preferred embodiments the MOG antigen comprises an amino acid sequence selected from the group consisting of amino acids 1-22, 34-56, 64-49 and 35-55 of SEQ ID NO: 18.

According to still further features in the described preferred embodiments the viral display vehicle comprises a filamentous bacteriophage.

According to still further features in the described preferred embodiments the filamentous bacteriophage is an fd bacteriophage.

According to still further features in the described preferred embodiments the filamentous bacteriophage comprises 150 copies of the antigen.

According to still further features in the described preferred embodiments the filamentous bacteriophage comprises 3000 copies of the antigen.

According to still further features in the described preferred embodiments the filamentous bacteriophage is selected from the group consisting of an M13 bacteriophage and an f1 bacteriophage.

According to still further features in the described preferred embodiments the multiple sclerosis associated antigen is selected from the group consisting of a myelin basic protein (MBP) antigen, a proteolipid protein (PLP) antigen, a myelin associated glycoprotein (MAG) antigen, a myelin-associated oligodendrocytic basic protein (MOBP) and an oligodendrocyte-specific protein (OSP).

According to still further features in the described preferred embodiments the therapeutically effective amount being capable of inducing immune tolerance.

The present invention successfully addresses the shortcomings of the presently known configurations by providing viral display vehicles displaying multiple sclerosis associated autoantigens which can be used for treating multiple sclerosis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of the biotechnology and medical arts.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic presentation depicting phage immunization protocols. Three groups of 8 weeks-old female C57BLU6 mice, were intranasally treated nine times with 25 μl of 5×10¹³ phages per ml phage constructs: phage alone; phage displaying MOG 150 copies (MOG 88), and phage displaying 3000 copies of MOG (MOG 8). The fourth group was treated with PBS. The first five administrations were performed prior to EAE induction every 3 days over a period of two weeks. Two weeks later, the mice were EAE induced (on days 30 or 31 of the experiment); on the next day the sixth administration was given. The following administrations were applied on day 45 (15 days after EAE induction; 7^{th} administration), on day 114 (84 days after EAE induction; 8^{th} administration) and on day 130 (100 days after EAE induction; the 9^{th} administration) of the experiment. Bleedings of the mice were performed before the first immunization, after the fifth immunization, between the 7^{th} and 8^{th} immunization (one month after EAE induction) and after the 9^{th} immunization (4 months after EAE induction).

FIGS. 2a-d depict EAE scores of mice subjected to the various immunization protocols depicted in FIG. 1. Mice (three in each group) were immunized with either wild-type phage (which did not include the MOG antigen) (FIG. 2b), pliages expressing MOG f88 (150 copies of MOG antigen) (FIG. 2c), phages expressing MOG f8 (3000 copies of MOG antigen) (FIG. 2d) or PBS (FIG. 2a), and following EAE induction the mice were observed for EAE induced phenotype using the EAE score test. Mice were observed daily for weight loss and clinical signs of EAE and scored on a scale 0-5 according to: 0, no disease; 1, limp tail; 2, hind limb weakness; 3, total hind leg or partial hind and front leg paralysis; 4, total hind leg and front leg paralysis; 5, moribund or dead. Note that while in mice treated with wild-type phage or PBS the EAE scores reached the levels of 3-5 even after 10 days of EAE induction, the EAE scores of mice treated with the MOG f88 (except for mouse 503) or the MOG f8 vectors remained as low as 0-1 for at least 150 days. Thus, these results demonstrate that immunization of mice with the viral display vehicle of the present invention which displays the MOG autoantigen is highly efficient in preventing EAE phenotype.

FIGS. 3a-b depict experimental design (FIG. 3a) and EAE scores (FIG. 3b) of mice treated with the phage MOG f88 displaying MOG₃₇₋₄₄. Mice were EAE induced using the MOG₃₅-55 emulsion and were further subjected to eight intranasal administrations (every three days) of either the phage MOG f88 (squares; f88 after induction) or PBS (triangles; EAE only). Note the significant effect of the viral display vehicle on ameliorating EAE induced symptoms (f88 after induction) as compared to the severe EAE symptoms (high EAE clinical scores) of mice subjected to EAE and administered with PBS alone (EAE only).

FIGS. 4a-c schematically depict maps of the phage display vectors of the present invention (FIGS. 4a-b) and a partial sequence alignment between the MOG-phage fusion constructs. FIG. 4a—The fd-tet phage f8-1 vector displaying MOG37-44 peptide fused to all 3,000 copies of the major coat protein pVIII. Peptide was cloned into PstI and BamHI restricted sites of a single copy of pVIII gene; FIG. 4b—The fd-tet phage f88-4 vector displaying MOG37-44 peptide fused to 150 copies of the pVIII by cloning into HindIII and PstI sites of a duplicated copy of pVIII gene that is regulated by tac promoter. FIG. 4c-Nucleic acid sequence alignment between partial sequences of MOG f8 (SEQ ID NO:22) and MOG f88 (SEQ ID NO:17) obtained from sequencing of positive clones containing the MOG epitope. The nucleic acid encoding the MOG epitope is set forth by (SEQ ID NO:12) and the encoded MOG₃₇₋₄₄ epitope amino acid sequence (VGWYRSPF) is set forth by SEQ ID NO:10.

### DESCRIPTION OF THE PREFERRED EMBODIMENTS

The present invention is of viral display vehicles displaying multiple sclerosis associated autoantigens on the surface thereof. Specifically, the present invention can be used to treat multiple sclerosis in a subject.

The principles and operation of the compositions and methods according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Multiple sclerosis (MS) is a chronic inflammatory disease of the central nervous system (CNS) characterized by a prominent infiltration of macrophages and T lymphocytes to the brain and spinal cord resulting in gliotic scars, axonal loss and demyelination in the brain and spinal cord. The disease develops in genetically predisposed subjects exposed to yet undefined environmental factors and the pathogenesis involves autoimmune mechanisms associated with autoreactive T cells against myelin antigens. Current therapy of MS involves the use of immunomodulatory drugs such as interferon beta-1a, however, such drugs have limited efficacy.

Attempts to treat MS by inducing tolerance against MS associated antigens include intranasal administration of encephalitogenic epitopes of MBP (Bai et al. 1997; Friedman and Weiner 1994; U.S. Pat. No. 5,645,820 to Hafler D A. and Weiner H L.). However, due to the limited effect on disease symptoms (e.g., only a slight decrease in EAE scores from 3-4 to 2; U.S. Pat. No. 5,645,820) such immunization modes are not clinically practiced.

The use of viral display vectors for immunization was demonstrated against diseases associated with formation of amyloid plaques such as Alzheimer's disease (U.S. Pat. No. 6,703,015 to Solomon B. and Frenkel D.). These vectors were designed to induce antibodies against a beta-amyloid epitope within the treated subject and thus treat Alzheimer's disease. However, since administration of the viral display vehicles resulted in production of endogenous antibodies against the displayed epitope, such display vehicles were never suggested for treating multiple sclerosis or any other autoimmune disease where auto-antibodies are deleterious and undesired.

While reducing the present invention to practice, the present inventors have uncovered a highly efficient method of treating multiple sclerosis by administering viral display vehicles displaying multiple sclerosis associated autoantigens on the surface thereof.

As described in Example 1 of the Examples sections which follows, the present inventors have constructed viral display vehicles of bacteriophage fd which display various copies of the MOG₃₇₋₄₄ amino acid sequence, a multiple sclerosis associated antigen. These compositions were able to prevent disease progression in a multiple sclerosis animal model (the EAE induced mouse) and to ameliorate symptoms of a full blown disease.

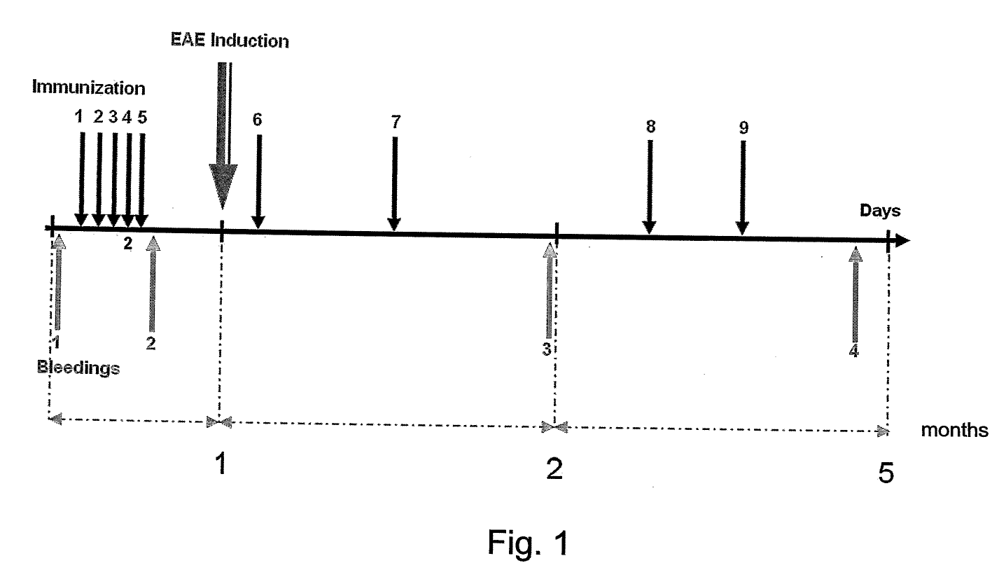

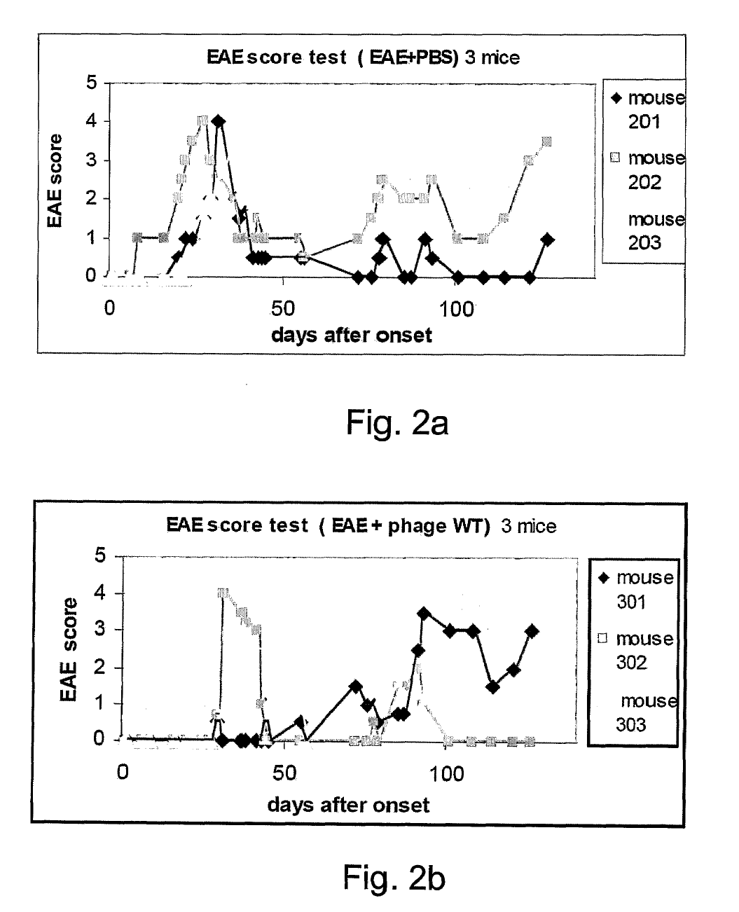

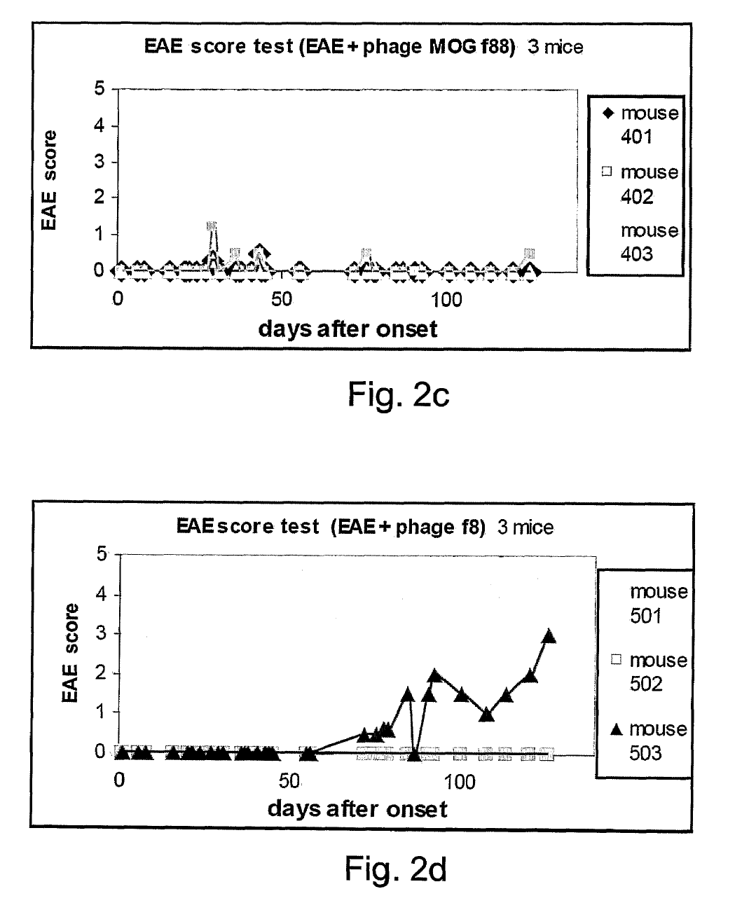

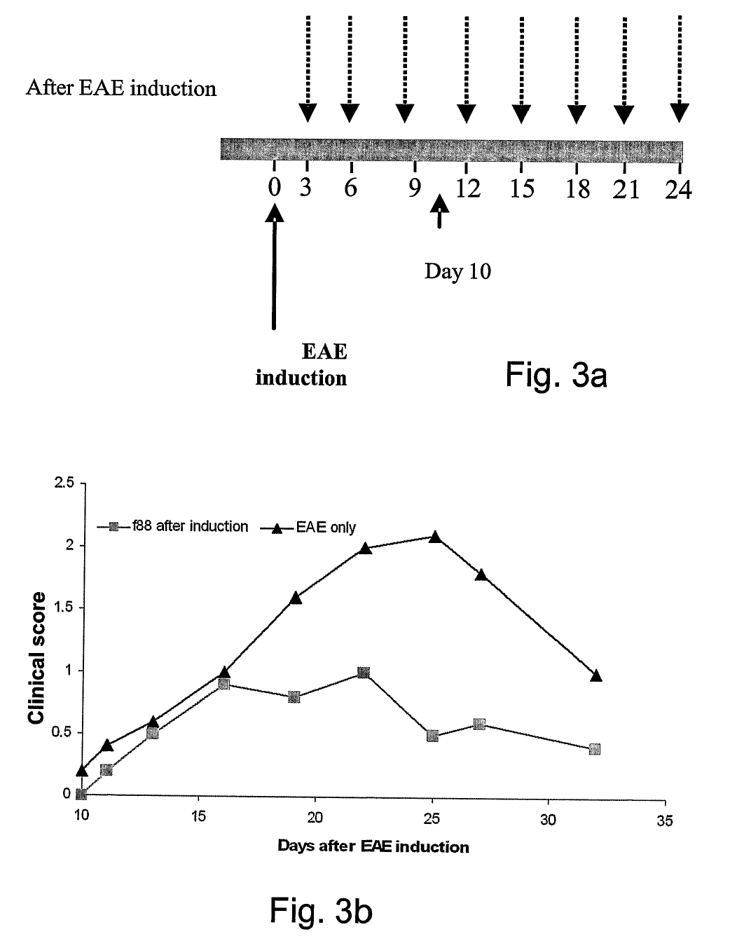

As is illustrated hereinbelow and in the Examples section which follows, while intraperitoneal administration of phage MOG f8 produced certain IgG antibodies titers, no IgG antibodies were observed following intranasal administration of the same phage. In addition, as is shown in FIGS. 1 and 2a-d and described in Example 1 of the Examples section which follows, serial intranasal immunizations of mice with either the MOG-f8 or MOG-f88 phages prior to and following EAE induction resulted in a complete abolishment of EAE phenotype for at least 150 days. In contrast, mice intranasally immunized with either PBS or an empty viral display vehicle (negative control) developed a sever EAE phenotype immediately after EAE induction (by subcutaneous injection with MOG35-55 peptide emulsified with incomplete Freund's adjuvant) which was maintained to various extents for at least 120 days. Moreover, as is shown in FIGS. 3a-b and described in Example 2 of the Examples section which follows, the MOG-f88 viral display vehicle was capable of ameliorating EAE-induced symptoms following disease onset. These results demonstrate the high therapeutic capacity of viral display vehicles displaying multiple sclerosis associated autoantigens to induce tolerance against the autoantigens and to treat multiple sclerosis.

Thus, according to one aspect of the present invention there is provided a composition-of-matter comprising a viral display vehicle displaying a multiple sclerosis associated autoantigen on a surface thereof.

As used herein the phrase “viral display vehicle” refers to any double stranded DNA viral particle, single stranded DNA viral particle or RNA viral particle capable of displaying the multiple sclerosis associated autoantigen of the present invention as a fusion protein of the virus coat protein.

Preferably, the viral display vehicle of the present invention comprises a filamentous bacteriophage. Such a filamentous phage is suitable for intranasal administration.

Non-limiting examples of viral display vehicles which can be used according to this aspect of the present invention include the fd bacteriophage, the M13 bacteriophage and the f1 bacteriophage.

The coat protein in which the autoantigen of the present invention is inserted (as a fusion protein) is preferably presented in multiple copies in each phage particle. Such a coat protein can be, for example, the major coat protein VIII (pVIII). Since the autoantigen is integrated in the viral coat protein, the number of copies of the coat protein reflects the number of copies of the autoantigen in each viral particle.

For example, as is shown in Example 1 of the Examples section which follows, the filamentous fd bacteriophage used by the present invention includes either 150 copies or 3000 copies of the coat protein VIII, thus, following ligation of the coding sequence encoding the autoantigen of the present invention into the coding sequence of the viral coat protein, the viral particle displays 150 or 3000 copies, respectively, of the autoantigen on the surface thereof.

Methods of constructing a viral display vehicle which displays the autoantigen of the present invention are well known in the art and are further described in the Example section which follows. Briefly, the coding sequence of the autoantigen of the present invention [e.g., SEQ ID NO:21 (5′-GTGGGGTGGTACCGCCCCCCCTTC-3′) which encodes the human MOG37-44 epitope (SEQ ID NO:19) is ligated in-frame (using recombinant DNA technologies) into the genomic sequence of the viral coat protein such that following expression of the coat protein by the viral particle the autoantigen of the present invention is presented (displayed) on the coat protein.

For example, as described under the “General Materials and Experimental Methods” and Example 1 of the Examples section which follows, the nucleic acid sequence (SEQ ID NO:12; 5′-GTGGGCTGGTATCGCAGTCCGTTT-3′) encoding the mouse multiple sclerosis antigen MOG₃₇₋₄₄ (SEQ ID NO: 10) was ligated into the pVIII rec coat protein of the vector phage f8 [GenBank Accession No. AF218734 (SEQ ID NO:11)] or of the vector phage f88 [GenBank Accession No. AF218363 (SEQ ID NO:20)].

As used herein the term “autoantigen” refers to an amino acid sequence of an endogenous protein of a subject which is capable of eliciting an immune response in the subject.

Non-limiting examples of multiple sclerosis associated autoantigens include amino acid sequences of the myelin basic protein (MBP) (e.g., amino acids 84-103, 80-99, 83-99, 85-99 of GenBank Accession No. NP_{—}001020272; SEQ ID NO:8), proteolipid protein (PLP) (e.g., amino acids 96-117, 106-125, 140-156 of GenBank Accession No. NP_{—}000524.3; SEQ ID NO:15), myelin-associated glycoprotein (MAG) (e.g., amino acids 20-34, 124-137, 354-377, 570-582 of GenBank Accession No. NP_{—}542167.1; SEQ ID NO: 16), myelin-associated oligodendrocytic basic protein (MOBP) (e.g., amino acids 21-39, 15-36, of GenBank Accession No. NP_{—}891980.1; SEQ ID NO:13), oligodendrocyte-specific protein (OSP) (e.g., amino acids 55-80, 179-207, 55-66 and 94-207 of GenBank Accession No. NP_{—}005593.2; SEQ ID NO:14), and myelin oligodendrocyte glycoprotein (MOG) [e.g. amino acids 1-22, 34-56, 37-44 and 64-96 of SEQ ID NO:7 (mouse MOG; GenBank Accession No. AAA03180); which correspond to amino acids 1-22, 34-56, 37-44 and 64-96 of SEQ ID NO: 18 (human MOG; GenBank Accession No. AAB08090), respectively [for MOG homology between human and mouse see Johns, T. G. and C. C. Bernard (1999), The structure and function of myelin oligodendrocyte glycoprotein. J. Neurochem. 72 (1): 1-9].

Preferably, the multiple sclerosis associated antigen used by the present invention comprises amino acids 37-44 of SEQ ID NO:18 (VGWYRPPF; SEQ ID NO:19).

Multiple sclerosis associated autoantigens can be identified from a plurality of synthetic peptides derived from a candidate protein (e.g., MOBP). Such peptides, can be, for example, overlapping peptides of 10-20 amino acids which are preferably emulsified in complete Freund's adjuvant (CFA) and further administered (e.g., intraperitoneally) into a multiple sclerosis animal model [the allergic encephalomyelitis (EAE) animal model; C57b1/6 mice (Shao H, Huang Z, Sun S L, Kaplan H J, Sun D. Myelin/oligodendrocyte glycoprotein-specific T-cells induce severe optic neuritis in the C57BL/6 mouse. Invest Opthalmol. Vis. Sci. 2004, 45: 4060-5)]. The effect of the administered peptides on EAE symptoms (as assessed using EAE acceptable scores, see e.g., the Examples section which follows) is evaluated and the candidate autoantigens are those peptides resulting in relatively high EAE scores (e.g., scores of 4-5), essentially as described in Holz A, et al., 2000 [J. Iimmunol. 164(2): 1103-9]. Candidate peptides are further qualified for their ability to induce an immune response in multiple sclerosis patients using, for example, a lymphocyte proliferation assay with lymphocytes obtained from multiple sclerosis patients [see for example, Holz, 2000 (Supra)].

It will be appreciated that the viral display vehicles which display the multiple sclerosis associated autoantigens of the present invention can be used to treat multiple sclerosis.

As used herein the phrase “treating” refers to inhibiting, preventing or arresting the development of a pathology (i.e., multiple sclerosis) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” (or “individual” which is interchangeably used herein) refers to an animal subject e.g., a mammal, e.g., a human being at any age who is diagnosed with or is at risk of developing the pathology. Non-limiting examples of individuals who are at risk to develop the pathology of the present invention include individuals who are genetically predisposed to develop the pathology (e.g., individuals who carry a mutation or a DNA polymorphism which is associated with high prevalence of the pathology), and/or individuals who are at high risk to develop the pathology due to presence of similar pathologies or other factors such as environmental hazard. For example, an individual who is diagnosed with a certain autoimmune disease (e.g., type I diabetes mellitus) is at higher risk of developing multiple sclerosis (see Janice S. et al., Type 1 Diabetes and Multiple Sclerosis: Together at last, Diabetes Care 26:3192-3193, 2003).

Thus, according to another aspect of the present invention there is provided a method of treating multiple sclerosis. The method is effected by administering to a subject in need thereof a therapeutically effective amount of the composition-of-matter of the present invention (the viral display vehicle which displays the multiple sclerosis associated autoantigen of the present invention), thereby treating multiple sclerosis.

As used herein a “therapeutically effective amount” refers to an amount of the composition-of-matter of the present invention (the viral display vehicle which displays the multiple sclerosis associated autoantigen of the present invention) which is capable of the biological effect (treating multiple sclerosis). Preferably, the therapeutically effective amount of the composition-of-matter of the present invention is selected such that it is capable of inducing immune tolerance against the autoantigens associated with multiple sclerosis while avoiding endogenous antibody production against the displayed autoantigens.

Thus, the viral display vehicle of the present invention which displays the multiple sclerosis associated autoantigens is preferably administered to oral or mucosal tissues where it is capable of inducing immune tolerance while avoiding production of autoantigens against multiple sclerosis. In case viral amplification within the subject (e.g., bacterial mediated) is less desired, measures may be taken to avoid contact with the natural flora (e.g., by mode of administration) or propagation therewith (e.g., UV radiated particles).

Oral and mucosal tolerance for suppression and treatment of autoimmune disease is known in the art. For example, Weiner et al. have disclosed therapy, for the treatment of rheumatoid arthritis by mucosal administration of collagen and collagen peptides (U.S. Pat. Nos. 5,399,347; 5,720,955; 5,733,542; 5,843,445; 5,856,446; and 6,019,975), treatment of Type I diabetes by mucosal administration of insulin (U.S. Pat. Nos. 5,643,868; 5,763,396; 5,843,445; 5,858,968; 6,645,504; and 6,703,361) or glucagon (U.S. Pat. No. 6,645,504), uveoretinitis by mucosal administration of toleragens (U.S. Pat. No. 5,961,977), and multiple sclerosis by mucosal administration of myelin basic protein (MBP) (U.S. Pat. Nos. 5,849,298; 5,858,364; 5,858,980; 5,869,093; 6,077,509). Additional candidate conditions, antigens and modes of treatment by mucosal tolerance have been disclosed in U.S. Pat. Nos. 6,812,205, 5,935,577; 5,397,771; 4,690,683 to Weiner et al., U.S. Pat. No. 6,790,447 to Wildner et al; International Patent Nos. EP 0886471 A1, WO 01821951 to Haas, et al, U.S. Pat. No. 5,843,449 to Boots et al. (HCgp-39 for arthritis), and U.S. patent application Ser. No. 10/437,404 to Das (mucosal tolerance and relief from Crohn's disease by administration of Colonic Epithelial Protein).

Induction of mucosal tolerance (e.g., using trans-mucosal administration) according to the invention is an advantageous method for treating multiple sclerosis for several reasons:

(1) Absence of toxicity: no toxicity has been observed in clinical trials or animal experiments involving oral or other mucosal administration of protein antigens, such as bovine myelin [which contains myelin basic protein (MBP) and proteolipid protein (PLP)] to humans afflicted with multiple sclerosis, or oral or by-inhalation administration of chicken Type II collagen to humans or rodents afflicted with rheumatoid arthritis [or a corresponding animal model disorder]; or oral administration of bovine S-antigen to humans afflicted with uveoretinitis; or oral administration of insulin to healthy volunteers.

(2) Containment of immunosuppression. Conventional treatments of immune system disorders involve administration of non-specific immunosuppressive agents, such as the cytotoxic drugs methotrexate, cyclophosphamide (CYTOXAN®, Bristol-Myers Squibb), azathioprine (IMURAN®, Glaxo Wellcome) and cyclosporin A (SANDIMMUNE®, NEORAL®, Novartis). Steroid compounds such as prednisone and methylprednisolone (also non-specific immunosuppressants) are also employed in many instances. All of these currently employed drugs have limited efficacy (e.g., against both cell-mediated and antibody-mediated autoimmune disorders). Furthermore, such drugs have significant toxic and other side effects and, more important, eventually induce “global” immunosuppression in the subject being treated. Prolonged treatment with the drugs down-regulates the normal protective immune response against pathogens, thereby increasing the risk of infection. In addition, patients subjected to prolonged global immunosuppression have an increased risk of developing severe medical complications from the treatment such as malignancies, kidney failure and diabetes.

(3) Convenience of therapy. Mucosal administration is more convenient than parenteral, or other forms, of administration.

(4) Greatly reduced incidence of alteration of the tolerizing molecule by digestive and metabolic processes (especially in non-oral routes of administration). These advantages provide superior protection from atherogenic processes, improved patient compliance and reduced cost of therapy.

Without being bound to any theory, induction of tolerance via oral or mucosal administration can result from interaction between the tolerizing autoantigen displayed on the viral display vehicle of the present invention and the mucosal associated lymphatic tissue (MALT), allowing the accumulation of tolerizing amounts of the autoantigen in the MALT.

Thus, induction of tolerance against the multiple sclerosis associated autoantigen is preferably performed by administering the composition-of-matter of the present invention to a mucosal surface of the subject.

As used herein, the phrase “mucosal surface” is defined as a portion of the anatomy having exposed mucosal membranes having component or components of the mucosal associated lymphatic tissue. As used herein, the phrase “mucosal administration” is defined as application of the composition-of-matter of the present invention to at least one mucosal surface. Non-limiting examples of mucosal administration are buccal, intranasal, otic (middle ear), conjunctival, vaginal, rectal, eye, etc. Mucosal administration excludes, for example, intravenous, subcutaneous and epidural administration.

It will be appreciated that the composition-of-matter of the present invention (which includes the viral display vehicle of the present invention which displays the autoantigen of the present invention) can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the composition-of-matter of the present invention (the viral display vehicle of the present invention which displays the multiple sclerosis associated autoantigen of the present invention) accountable for the biological effect (treating multiple sclerosis).

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable formulations according to the invention include formulations of the composition-of-matter of the present invention adapted for oral, enteral, buccal, nasal, bronchial or intrapulmonary administration. The preparation of such formulations is well within the skill of the art. Thus, it is preferred that such formulations not contain substances that can act as adjuvants in order to avoid sensitization of the treated subject.

Suitable oral formulations for use according to the present invention can be in any suitable orally administrable form, for example, a pill, a liquid, or a capsule or caplet containing an effective amount of the autoantigen. Each oral formulation may additionally comprise inert constituents including pharmaceutically acceptable carriers, diluents, fillers, disintegrants, flavorings, stabilizers, preservatives, solubilizing or emulsifying agents and salts as is well-known in the art. For example, tablets may be formulated in accordance with conventional procedures employing solid carriers and other excipients well-known in the art. Capsules may be made from any cellulose derivatives. Non-limiting examples of solid carriers include starch, sugar, bentonite, silica and other commonly used inert ingredients. Diluents for liquid oral formulations can include inter alia saline, syrup, dextrose and water.

The composition-of-matter of the present invention (which includes the viral display vehicle displaying the autoantigen of the present invention) can also be made up in liquid formulations or dosage forms such as, for example, suspensions or solutions in a physiologically acceptable aqueous liquid medium. Such liquid media include water, or suitable beverages, such as fruit juice or tea which will be convenient for the patient to sip at spaced apart intervals throughout the day. When given orally in liquid formulations the composition-of-matter of the present invention may be dissolved or suspended in a physiologically acceptable liquid medium, and for this purpose the composition-of-matter of the present invention may be solubilized or adjusted to a pH within physiologically acceptable limits (e.g., 3.5 to 8).

Sustained release oral delivery systems are also contemplated and are preferred. Non-limiting examples of sustained release oral dosage forms include those described in U.S. Pat. No. 4,704,295, issued Nov. 3, 1987; U.S. Pat. No. 4,556,552, issued Dec. 3, 1985; U.S. Pat. No. 4,309,404, issued Jan. 5, 1982; U.S. Pat. No. 4,309,406, issued Jan. 5, 1982; U.S. Pat. No. 5,405,619, issued Apr. 10, 1995; PCT International Application WO 85/02092, published May 23, 1985; U.S. Pat. No. 5,416,071, issued May 16, 1995; U.S. Pat. No. 5,371,109, issued Dec. 6, 1994; U.S. Pat. No. 5,356,635, issued Oct. 18, 1994; U.S. Pat. No. 5,236,704, issued Aug. 17, 1993; U.S. Pat. No. 5,151,272, issued Sep. 29, 1992; U.S. Pat. No. 4,985,253, issued Jan. 15, 1991; U.S. Pat. No. 4,895,724, issued Jan. 23, 1990; and U.S. Pat. No. 4,675,189, issued Jun. 23, 1987, incorporated as if fully set forth herein by reference.

Sustained release oral dosage forms coated with bioadhesives can also be used. Examples are compositions disclosed in European Published Application EP 516141; U.S. Pat. No. 4,226,848; U.S. Pat. No. 4,713,243; U.S. Pat. No. 4,940,587; PCT International Application WO 85/02092; European Published Application 205282; Smart J D et al. (1984) J Pharm Pharmacol 36:295-9; Sala et al. (1989) Proceed Intern Symp Control Rel Bioact Mater 16:420-1; Hunter et al. (1983) International Journal of Pharmaceutics 17:59-64; “Bioadhesion—Possibilities and Future Trends, Kellaway,” Course No. 470, May 22-24, 1989, incorporated as if fully set forth herein by reference.

Commercially available sustained release formulations and devices include those marketed by ALZA Corporation, Palo Alto, Calif., under tradename ALZET, INFUSET, IVOS, OROS, OSMET, or described in one or more U.S. Pat. No. 5,284,660, issued Feb. 9, 1994; U.S. Pat. No. 5,141,750, issued Aug. 25, 1992; U.S. Pat. No. 5,110,597, issued May 5, 1992; U.S. Pat. No. 4,917,895, issued Apr. 17, 1990; U.S. Pat. No. 4,837,027, issued Jun. 6, 1989; U.S. Pat. No. 3,993,073, issued Nov. 23, 1976; U.S. Pat. No. 3,948,262, issued Apr. 6, 1976; U.S. Pat. No. 3,944,064, issued Mar. 16, 1976; and U.S. Pat. No. 3,699,963; International Applications PCT/US93/10077 and PCT/US93/11660; and European Published Applications EP 259013 and EP 354742, incorporated as if fully set forth herein by reference.

Orally administrable pharmaceutical formulations containing the composition-of-matter of the present invention are prepared and administered to mammals who have manifested symptoms of multiple sclerosis. Additionally, subjects who are at risk for developing multiple sclerosis, e.g., having a genetic predisposition to developing the disorder, as determined through suitable means, such as genetic studies and analysis, are treated with similar oral preparations.

Pharmaceutical formulations for oral or enteral administration to treat multiple sclerosis are prepared from the composition-of-matter of the present invention and a pharmaceutically acceptable carrier suitable for oral ingestion.

For by-inhalation administration (i.e., delivery to the bronchopulmonary mucosa) suitable sprays and aerosols can be used, for example using a nebulizer such as those described in U.S. Pat. No. 4,624,251 issued Nov. 25, 1986; U.S. Pat. No. 3,703,173 issued Nov. 21, 1972; U.S. Pat. No. 3,561,444 issued Feb. 9, 1971; and U.S. Pat. No. 4,635,627 issued Jan. 13, 1971, incorporated as if fully set forth herein by reference. The aerosol material is inhaled by the subject to be treated.

Other systems of aerosol delivery, such as the pressurized metered dose inhaler (MDI) and the dry powder inhaler as disclosed in Newman S P in Aerosols and the Lung, S W Clarke S W and D Davis, eds. pp. 197-224, Butterworths, London, England, 1984, can be used when practicing the present invention.

Aerosol delivery systems of the type disclosed herein are available from numerous commercial sources including Fisons Corporation (Bedford, Mass.), Schering Corp. (Kenilworth, N.J.) and American Pharmoseal Co. (Valencia, Calif.).

Formulations for nasal administration can be administered in an aqueous solution. Preferred aerosol pharmaceutical formulations may comprise for example, a physiologically acceptable buffered saline solution containing the composition-of-matter of the present invention.

Specific non-limiting examples of the carriers and/or diluents that are useful in the pharmaceutical formulations of the present invention include water and physiologically acceptable buffered saline solutions such as phosphate buffered saline solutions pH 7.0-8.0.

The mucosally administered formulation of the present invention may include a thermosetting gel which increases in viscosity at body temperature upon contact with the mucosa.

Formulations for buccal administration can include mucoadhesive mixed with effective amounts of the composition-of-matter of the present invention. Effective amounts are anticipated to vary according to the formulation employed. For formulation administered by inhalation, the effective amount is likely to be less than that of the oral dose.

The treatment may be discontinued if desired (in the judgment of the attending physician) and the patient monitored for signs of relapse. If clinical symptoms or other disorder indicators show that the patient is relapsing, treatment may resume.

As will be understood by those skilled in the art, the dosage will vary with the various compositions-of-matter of the present invention and may vary with the sex, age, and physical condition of the patient as well as with other concurrent treatments being administered. Consequently, adjustment and refinement of the dosages used and the administration schedules will preferably be determined based on these factors and especially on the patient's response to the treatment. Such determinations, however, require no more than routine experimentation, as illustrated in Examples provided below.

Determination of a therapeutically effective amount is well within the capability of those skilled in the art, especially in light of the detailed disclosure provided herein.

For any preparation used in the methods of the invention, the toxicity, therapeutically effective amount or dose can be estimated initially from in vivo animal models. The data obtained from these in vitro or animal studies can be used in formulating a range of dosage for use in human. For example, a dose can be formulated in animal models [e.g., the EAE mouse model for multiple sclerosis, C57b1/6 mice (Shao H, Huang Z, Sun S L, Kaplan H J, Sun D. Myelin/oligodendrocyte glycoprotein-specific T-cells induce severe optic neuritis in the C57BL/6 mouse. Invest Opthalmol V is Sci. 2004, 45: 4060-5] to achieve a desired concentration or titer. Such information can be used to more accurately determine useful doses in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient (the composition-of-matter of the present invention) which are sufficient to induce tolerance against the autoantigen and treat multiple sclerosis (minimal effective concentration, MEC). The MEC will vary for each preparation, but can be estimated from in vitro/in vivo data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

It will be appreciated that the composition-of-matter of the present invention can be provided to the individual along with other known multiple sclerosis agents such as interferon beta-1a in order to increase the therapeutic effect thereof. However, measures are taken to avoid cross-effectiveness and unwanted side effects.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Compositions of the present invention may be included in an article-of-manufacture, packaged and identified for use in the treatment of multiple sclerosis in a subject in need thereof. The article-of-manufacture includes a packaging material and the composition-of-matter of the present invention. The packaging material including a label or package insert indicating that the composition-of-matter of the present invention is for treating multiple sclerosis.

It is expected that during the life of this patent many relevant viral display vehicles will be developed and the scope of the term viral display vehicle is intended to include all such new technologies a priori.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Construction of a phage vector displaying the MOG 37-44 amino acid sequence (SEQ ID NO:10)

Preparation of competent E. coli cells—E. coli K91 Kan were grown in 2 ml of 2YT media containing 100 μg/ml Kanamycin at 37° C. while shaking (250 RPM) overnight. Then, 500 μl of overnight culture was transferred to 100 ml SOB media grown to an early logarithmic phase [O.D. (600 nm)˜0.3]. The grown bacteria were incubated on ice for 10 minutes and then centrifuged at 5000 rpm for 10 minutes at 4° C. The pellet was resuspended in 20 ml CCMB and incubated for 20 minutes on ice followed by 10 minutes centrifugation at 5000 rpm at 4° C. The precipitant bacteria were resuspended in 4 ml CCMB and were kept on ice while divided into aliquots and stored at −70° C.

### Cloning of Phage MOG f8 and MOG f88

F8-1 vector purification and cleavage—The vector f8-1 was purified from a “type 8” fd bacteriophage library clone using a Qiagen DNA purification kit [Smith G P, Scott J K (1993) Libraries of peptides and proteins displayed on filamentous phage. Methods Enzymol 217:228-257; Smith G P (1991) Surface presentation of protein epitopes using bacteriophage expression systems. Curr Opin Biotechnol 2:668-673]. The vector was digested by two PstI and BamHI restriction enzymes separately. First, a total reaction volume of 40 μl containing 15 μg of dsDNA, 1 μl PstI, 4 μl of a suitable restriction enzyme buffer was incubated for 1 hour at 37° C., followed by inactivation at 70° C. for 5 minutes. BamHI (1 μl) was then added to the reaction mixture and was further incubated for 1 hour at 37° C. followed by inactivation at 70° C. for 5 minutes. In order to remove the 5′ phosphate groups from the digested vector, 1 μl of Antarctic Phosphatase was added for a 30-minutes incubation at 37° C. The phosphatase treated DNA was then applied onto 0.7% agarose gel electrophoresis. The linearized vector was purified from gel using Qiagen DNA extraction kit

F88-4 vector purification and cleavage—Purification and digestion of f88-4 vector, “type 88” was performed similarly to that described above (Smith, 1991, 1993, Supra), the difference being that HindIII and PstI were added together for 2 hour incubation at 37° C. Then the vector was de-phosphorylated and purified as explained above.

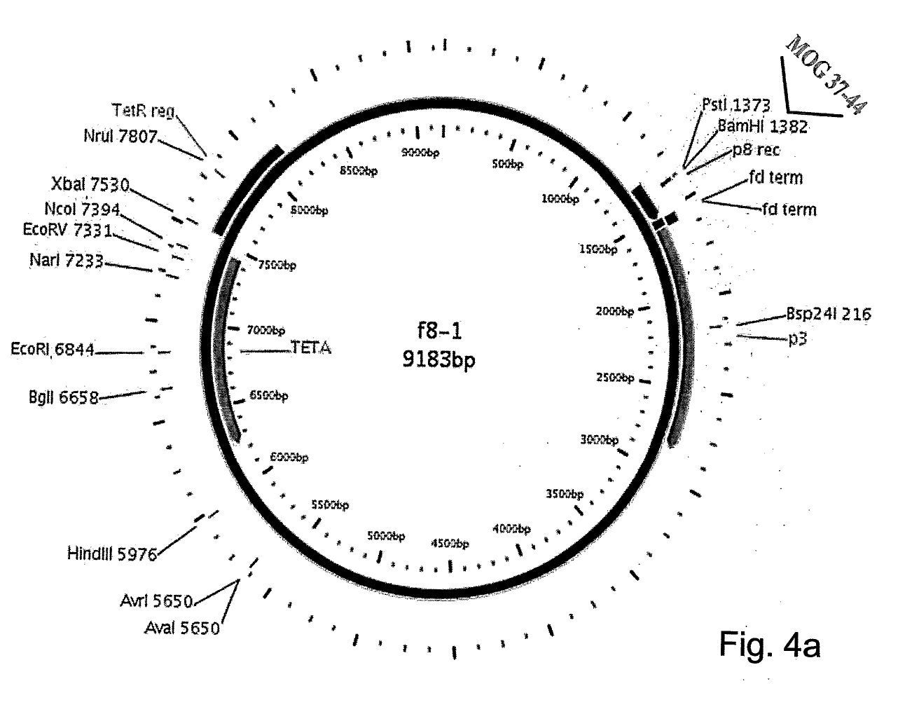

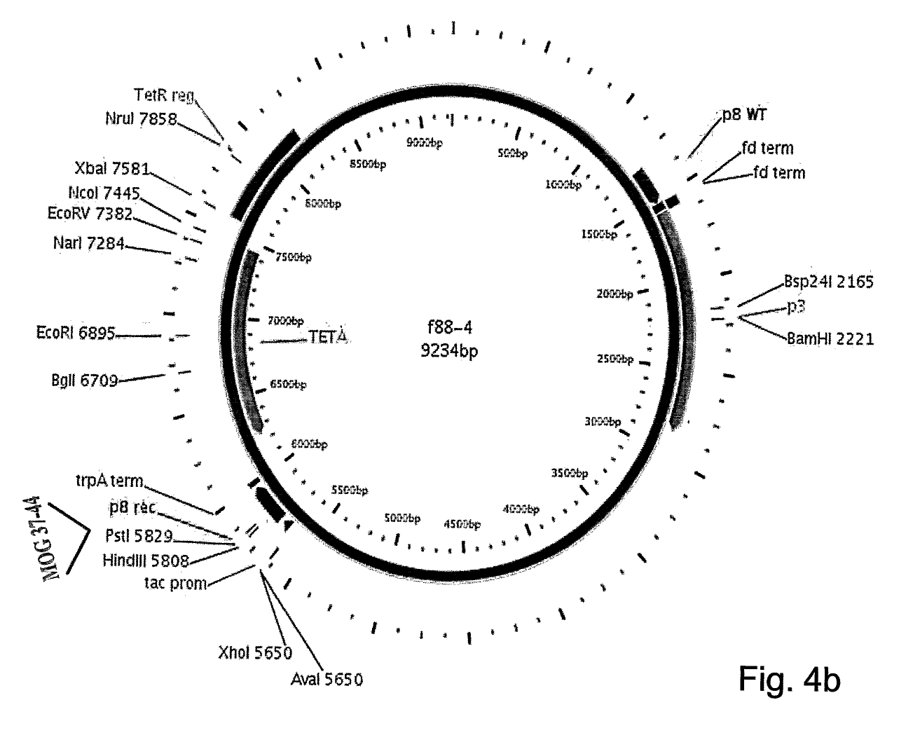

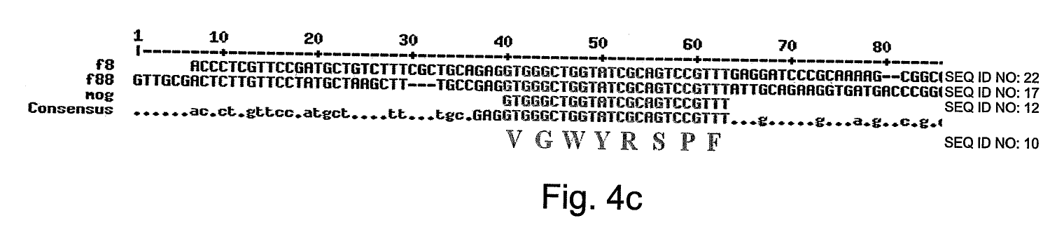

Insert preparation: Phosphorylation of insert primers and annealing—Two complementary primers were synthesized in Sigma, each primer separately; 5 μl of 10 μmol/μl was mixed with 2.5 μl T4 kinase bufferX10, 1 μl T4 kinase and 16.5 μl double distilled waster (DDW) followed by 2 hours incubation at 37° C. subsequently inactivated for 10 minutes at 70° C. Then, 20 μmol of each primer were added and incubated together for 5 minutes at 95° C., after which the temperature was gradually lowered. For the f8-1 vector, annealed insert primers [Forward primer (SEQ ID NO:1): 5′-GAGGTGGGCTGGTATCGCAGTCCGTTTGAG-3′; reverse primer (SEQ ID NO:2): 5′-GATCCTCAAACGGACTGCGATACCAGCCCACCTCTGCA-3′] results in dsDNA with PstI and BamHI sticky ends, and for the f88-4 annealed insert primers [Forward primer (SEQ ID NO:3): 5′-AGCTTTGCCGAGGTGGGCTGGTATCGCAGTCCGTTTATTGCA-3′; reverse primer (SEQ ID NO:4): 5′-ATAAACGGACTGCGATACCAGCCCACCTCGGCAA-3′] results in dsDNA with HindIII and PstI sticky ends (see vector maps in FIGS. 4a-b).

Ligation and transformation—Purified vector and annealed primers (insert) were ligated at a ratio of 1:3 respectively, as follows: 100 ng of vector, 1 ng insert, 1.5 μl DNA ligase buffer X10, 1 μl DNA ligase, incubated overnight at 16° C. The ligation products were transferred into competent K91Kan using heat shock method. 10 μl of ligation was added to 100 μl of bacteria and the mix was kept on ice for 30 minutes, followed by 2 minutes incubation at 42° C. then returned to ice. 2YT (1 ml) was added and bacteria were incubated by shaking at 150 rpm for 1.5 hours at 37° C. to express antibiotic resistant genes. Transformed cells were grown at 37° C. on 2YT plates containing 100 μg/ml Kanamycin and 20 μg/ml Tetracycline overnight.

Identification of positive clones—A number of colonies that had grown on plates were screened for the presence of insert using colony PCR. Each colony was mixed with 7 μl of ready mix, 5 μl sterile DDW, and 1 μl (10 μmol) of each primer and subjected to PCR reaction. The primers used were as follows: The forward primer of the insert had served as forward primer (i.e., SEQ ID NO: 1 served as a forward primer for identification of positive clones in f8 vector and SEQ ID NO:3 and served as a forward primer for identification of positive clones in f88 vector) and the reverse primer was complementary either to pVIII of f8-1 vector (5′-CAGCTTGCTTTCGAGATGA-3′; SEQ ID NO:5) or the f88-4 vector (5′-AGTAGCAGAAGCCTGAAGA-3′; SEQ ID NO:6). The PCR products were then applied to a 2% agarose gel in order to detect 170 bp bands indicating that the MOG insert is on the plasmid. Positive colonies were then sequenced using reverse primer that was used in PCR. FIG. 4c depicts partial sequence alignment of positive clones.

Scale-up of phage production—The cloned E. coli K91Kan containing vector f8 with MOG insert were grown in 500 ml of 2YT media, including 100 μg/ml Kanamycin and 20 μg/ml Tetracycline overnight at 37° C., to which K91Kan with vector f88 MOG with an addition of 2 mM IPTG was supplied. The next day, the inoculum was centrifuged at 6500 rpm at 4° C. for 20 minutes in order to eliminate bacteria presence. Supernatant was collected and incubated with PEG/NaCl in 5:1 (v/v) ratio overnight at 4° C. which enables phage precipitation. Phages were then precipitated by one hour centrifugation at 9000 rpm at 4° C. After supernatant was discarded, the precipitate was resuspended in 20 ml of sterile PBS and precipitated

$$\frac{O . D {.}_{( 269 nm )} - O . D {.}_{( 320 nm )} ⋆ 6 ⋆ {10}^{1}}{9273} = phages \text{/} ml$$

once again by overnight incubation with PEG/NaCl at ratio 1:5 at 4° C. Phage precipitation was achieved by a one hour centrifugation at 9000 rpm at 4° C., resuspended in 1 ml sterile PBS and then filtrated using 0.45 μm filter tip to eliminate any traces of bacteria. The recovered phage concentration was determined according to its absorbance at 269 nm and 320 nm as reference, measured by spectrophotometer, according to the formula:

EAE induction in mice—Female C57BL/6 mice, 10-weeks old, were immunized with myelin oligodendrocyte glycoprotein peptide [MOG35-55; MEVGWYRSPFSRVVHLYRNGK (SEQ ID NO:9)] 150 μg/200 μl, synthesized by PEPTIDES international company, Louisville, Ky., purified to 98.6% by HPLC. MOG35-55 peptide was dissolved in 100 μl double distilled water (DDW) and emulsified with additional 100 μl incomplete Freund's adjuvant (IFA) (Sigma-Aldrich, St. Louis, Mo.) containing 500 ng heat-inactivated H37Ra Mycobacterium tuberculosis (Difco, Detroit, Mich., USA). A total of 200 μl MOG emulsion was subcutaneously injected into four sites on the flanks of mice near the tail. At days 0 and 1 of post-immunization, mice received additional injections (intraperitoneally) of Pertussis Toxin (Sigma, Deisenhofen, Germany), 500 ng/300 μl PBS.

EAE scoring system—The phenotype of EAE induction was scored on a scale of 0-5 according to: “0”, no disease; “1”, limp tail; “2”, hind limb weakness; “3”, total hind leg or partial hind and front leg paralysis; “4”, total hind leg and front leg paralysis; “5”, moribund or dead.

### Example 1

### Intranasal Administration of a Viral Display Vehicle Displaying MOG Autoantigens Prevents EAE-Induced Phenotype

To test whether intranasal administration of a viral display vehicle displaying a MOG peptide can induce tolerance against multiple sclerosis associated autoantigens, the present inventors have challenged C57BL/6 mice with phage MOG-f8 or MOG-f88, as follows.

### Experimental Results

Construction of viral display vehicles displaying the MOG37-44 epitope (VGWYRSPF; SEQ ID NO:10)—The present inventors have genetically engineered a recombinant fd phage, displaying at its surface a chimeric pVIII major coat protein fused to the MOG₃₇₋₄₄ amino acid sequence (SEQ ID NO:10) which is part of the previously identified encephalogenetic peptide MOG₃₅₋₅₅ (MEVGWYRSPFSRVVHLYRNGK; SEQ ID NO:9). The recombinant phages MOG-f8 and MOG-f88 displayed 3000 or 150 copies of the MOG₃₇44 epitope, respectively. Expression of the MOG₃₇₋₄₄ epitope was measured by testing the reactivity of bacteriophages to polyclonal antibodies against peptide MOG₃₅₋₅₅ (SEQ ID NO:9). The positive results demonstrated that the recombinant phages displayed the 37-44 amino acid sequence of MOG on their surface.

Intranasal administration of phage MOG-f8 resulted in no IgG antibodies—In animals that received five doses of phage MOG-f8 intranasally, no IgG antibodies were detected against MOG₃₅₋₅₅, while intraperitoneal administration of mice produced certain titers against MOG₃₅₋₅₅.

Viral display vehicle displaying multiple sclerosis associated MOG autoantigen is capable of preventing development of EAE disease—The next step was to evaluate the ability of viral display vehicle of the present invention which displays the MOG37-44 antigen to prevent development of EAE disease. Briefly, 8 weeks-old female C57BL/6 mice were intranasally treated eight times with phage displaying 3000 copies of MOG (MOG 8) (25 μl of 5×10¹³ phages/ml) or phage displaying 150 copies of MOG (MOG 88) (25 μl of 5×10¹³ phages/ml). The first five treatments were given every 3 days (for a period of two weeks) prior to EAE induction. Two weeks later the mice were EAE induced using the MOG35-55 emulsion. The following four administrations were applied after 1 day (6^{th} administration), 2 weeks (7^{th} administration), 2.5 months (8^{th} administration) and 3.5 months (9^{th} administration) (FIG. 1). The mice were observed daily for clinical signs of EAE. As shown in FIGS. 2a-d, phage treatment before and after EAE induction prevented the disease progression.

These results demonstrate that intranasal administration of mice with the viral display vehicle of the present invention (e.g., phage MOG f8) is highly efficient in preventing EAE phenotype. Thus, these results suggest that intranasal administration of the viral display vehicle of the present invention which display a multiple sclerosis associated autoantigen (e.g., human MOG₃₇₋₄₄ as set forth by SEQ ID NO:19) can be used for preventing multiple sclerosis symptoms and thus treating multiple sclerosis.

### Example 2

### Treating of EAE-Induced Mice Using the Viral Display Vehicle Displaying a MOG Autoantigen

To further evaluate the capacity of the viral display vehicle of the present invention (which displays a multiple sclerosis associated antigen, e.g., MOG₃₇₋₄₄), the present inventors have intranasally administered the viral display vehicle to mice which were subjected to EAE induction with the MOG35-55 emulsion, as follows.

Eight weeks-old female C57BL/6 mice were subjected to EAE induction using the MOG35-55 emulsion and following EAE induction the mice were intranasally treated eight times with phage displaying 150 copies of MOG (MOG 88) (25 μl of 5×10¹³ phages/ml). Intranasal administrations of the MOG 88 were performed on days 3, 6, 9, 12, 15, 18, 21 and 24 following EAE induction (FIG. 3a). The mice were observed daily for clinical signs of EAE. As is shown in FIG. 3b, intranasal administration of the MOG 88 phage after EAE induction resulted in drastic amelioration of disease symptoms.

These results demonstrate that intranasal administration of mice with the viral display vehicle of the present invention which displays a multiple sclerosis associated autoantigen (e.g., phage MOG f88) is highly efficient in treating EAE symptoms. Thus, these results suggest the use of a viral display vehicle displaying a multiple sclerosis associated autoantigen such as human MOG37-44 (SEQ ID NO:19) for treating multiple sclerosis after disease onset (i.e., after multiple sclerosis was diagnosed in the subject).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

### REFERENCES

### (Additional References are Cited in Text)

1. Amor, S., N. Groome, C. Linington, et al. (1994). “Identification of epitopes of myelin oligodendrocyte glycoprotein for the induction of experimental allergic encephalomyelitis in SJL and Biozzi AB/H mice.” J Immunol 153(10): 4349-4356;
2. Bai, X.-F., F.-D. Shi, B.-G. Xiao, et al. (1997). “Nasal administration of myelin basic protein prevents relapsing experimental autoimmune encephalomyelitis in DA rats by activating regulatory cells expressing IL-4 and TGF-[beta] mRNA.” Journal of Neuroimmunology 80(1-2):65-75;
3. Friedman, A. and H. L. Weiner (1994). “Induction of Anergy or Active Suppression Following Oral Tolerance is Determined by Antigen Dosage.” PNAS 91(14): 6688-6692;
4. Kerlero de Rosbo, N., M. Hoffman, I. Mendel, et al. (1997). “Predominance of the autoimmune response to myelin oligodendrocyte glycoprotein (MOG) in multiple sclerosis: reactivity to the extracellular domain of MOG is directed against three main regions.” Eur J Immunol 27(11): 3059-69;
5. Pham-Dinh, D., M. Mattei, J. Nussbaum, et al. (1993). “Myelin/Oligodendrocyte Glycoprotein is a Member of a Subset of the Immunoglobulin Superfamily Encoded within the Major Histocompatibility Complex.” PNAS 90(17): 7990-7994;
6. Parkman, Graft-versus-host Disease, Ann. Rev. Med., 1991, 42: 189-197;
7. Sao H., et al., 2004. Invest. Ophthalmol. Vis. Sci. 45: 4060-4065;
8. Sun D., et al., 2001. The Journal of Immuunology 166: 7579-7587;

1. A composition-of-matter comprising a viral display vehicle displaying a multiple sclerosis associated antigen on a surface thereof.

2. A pharmaceutical composition comprising, as an active ingredient, the composition-of-matter of claim 1, and a pharmaceutically acceptable carrier.

3. A method of treating a multiple sclerosis, the method comprising administering to a subject in need thereof a therapeutically effective amount of the pharmaceutical composition of claim 2, thereby treating the multiple sclerosis.

4. (canceled)

5. The pharmaceutical composition of claim 2, wherein said pharmaceutically acceptable carrier is formulated for mucosal administration.

6. The method of claim 3, wherein said administering is effected by trans-mucosal administration.

7. The method of claim 3, wherein said administering is effected by intranasal administration.

8-9. (canceled)

10. The composition-of-matter of claim 1, wherein said multiple sclerosis associated antigen comprises a MOG antigen.

11. The composition-of-matter of claim 10, wherein said MOG antigen comprises amino acids 37-44 of SEQ ID NO:18.

12. The composition-of-matter of claim 10, wherein said MOG antigen comprises an amino acid sequence selected from the group consisting of amino acids 1-22, 34-56, 64-49 and 35-55 of SEQ ID NO:18.

13. The composition-of-matter of claim 1, wherein said viral display vehicle comprises a filamentous bacteriophage.

14. The composition-of-matter of claim 13, wherein said filamentous bacteriophage is an fd bacteriophage.

15. The composition-of-matter of claim 14, wherein said filamentous bacteriophage comprises 150 copies of said antigen.

16. The composition-of-matter of claim 14, wherein said filamentous bacteriophage comprises 3000 copies of said antigen.

17. The composition-of-matter of claim 13, wherein said filamentous bacteriophage is selected from the group consisting of an M13 bacteriophage and an fl bacteriophage.

18. The composition-of-matter of claim 1, wherein said multiple sclerosis associated antigen is selected from the group consisting of a myelin basic protein (MBP) antigen, a proteolipid protein (PLP) antigen, myelin associated glycoprotein (MAG) antigen, myelin-associated oligodendrocyte basic protein (MOBP) and oligodendrocyte-specific protein (OSP).

19. The method of claim 3, wherein said therapeutically effective amount is capable of inducing immune tolerance.
